(19) United States
(12) Patent Application Publication
(10) Pub. No.: US 2023/0125244 A1
(43) Pub. Date: Apr. 27, 2023

(54) APPARATUS AND METHODS FOR DECONTAMINATING ENCLOSED SPACES

(71) Applicant: ASEPTICK LIMITED, Andover, Hampshire (GB)

(72) Inventor: Nicholas Mark Turner ADAMS, Andover (GB)

(21) Appl. No.: 17/926,219

(22) PCT Filed: May 19, 2021
(86) PCT No.: PCT/GB2021/051211

§ 371 Date: Nov. 18, 2022

(30) Foreign Application Priority Data
May 19, 2020 (GB) 2007453.0

(51) Int. Cl. A61L 2/20

(52) U.S. Cl. CPC A61L 2/208; A61L 2202/11; A61L 2202/15; A61L 2202/25

(57) ABSTRACT

The invention relates to an apparatus for vapour decontamination of an enclosed space. The apparatus comprises: (a) a reservoir comprising a supply of a liquid decontaminant; and (b) a vaporiser unit in fluid communication with the reservoir. The vaporiser unit comprises: a body that defines a lumen that passes along the length of the body, the lumen comprising a first end and a second end, wherein the first end receives liquid decontaminant from the reservoir; and a heating element that is configured to deliver sufficient thermal energy to effect rapid boiling of the liquid decontaminant within the lumen so that it exits the lumen via an exit port at the second end of the lumen as a vapour. The exit port is configured to deliver the vapour into a flow of a non-heated carrier gas, thereby facilitating distribution of the decontaminant vapour within the enclosed space. The invention also relates to a method for decontaminating surfaces within an enclosed space.

### FIELD OF THE INVENTION

This invention relates to apparatus and methods for decontaminating enclosed spaces, including sealed enclosures such as rooms, chambers and environmental spaces as well as the surfaces and the equipment contained therein.

### BACKGROUND OF THE INVENTION

Global demand for systems that rapidly inactivate microbial contamination within enclosed environments has increased significantly with the growth in cell and gene therapies which require aseptic manufacturing facilities as well as the growing threat from pandemic viral infection, such as from influenza, Ebola, MERS and SARS, especially SARS-CoV-2. The rapid spread of COVID-19 disease in early-2020, as well as the severe economic damage resulting from the social distancing measures needed to control the infection, demonstrate that there is an increased need for decontamination methods and apparatus that are both reliable and straightforward to deploy.

Hydrogen peroxide vapour has been used to generate aseptic enclosures by the inactivation of micro-organisms (e.g. bacteria, virus and fungi) and has been used for over 20 years. Hydrogen peroxide vapour is attractive as a decontaminant as it can be readily catalytically broken down to water vapour and oxygen making it extremely environmentally friendly in comparison to conventional disinfectant chemicals such as chlorine- or quaternary ammonium-based biocides. It is also highly practical as there are no problematic chemical residues which could taint bio-pharmaceutical products or potentially poison patients or contaminate medical equipment in hospitals or contaminate food processing surfaces or retail sites.

As described by Watling in patents WO 01/21223 A1 and EP-1487503, an aqueous solution of hydrogen peroxide is directed via gravity, or using a pump, onto a heated surface such as a heated plate. The plate is heated to a temperature sufficient to facilitate flash evaporation of the hydrogen peroxide solution. Watling describes the necessity for flash evaporation to ensure that both the water and hydrogen peroxide components of the solution remain in the same ratios in the vapour phase as in the original liquid phase. Typically, flash evaporation occurs within a heated carrier gas stream, suitably air, which is delivered into the enclosure to be decontaminated. Watling describes in WO 01/21223 that the maximum concentration of vapour that can be delivered to the chamber depends, in part, on the temperature of the carrier gas stream entering the enclosure to be decontaminated. It is accepted by those skilled in the art that the carrier gas must be heated to allow sufficient hydrogen peroxide to be evaporated into the carrier gas and delivered to the chamber. This conventional “wisdom” is reflected in the presented art, including recent Italian patent publication ITMI20131246 (A1).

One of the problems that designers and manufacturers of hydrogen peroxide vapour generators face is that demand for the systems can include facilities, such as hospital wards or laboratories, where the electrical power supply is a standard supply. In other words, the application of the hydrogen peroxide vapour decontamination technology is limited if it needs to rely on “industrial” 3 phase power. Within Europe and much of Asia the “standard” electrical power supply is around 230V but is much lower in the United States and Canada (120V) as well as Japan (100V). As such, manufacturers and service-providers are limited by the quantity of power that is available to operate their hydrogen peroxide vapour systems and in particular the power requirements of the heaters in the system. Heating air, particularly rapidly moving air, requires large amounts of energy. This energy is wasted energy, as heating the atmosphere of the enclosed space is undesirable and is unhelpful for users of such systems in terms of minimising their carbon footprint. An example of this is the use of vapour generators in aseptic processing isolators, particularly those used to manipulate cell-based healthcare therapies (CBHTs). Often these cell-based therapies cannot be exposed to significantly increased temperatures as this will lead to increase in culture media temperature causing degradation of cells or other adverse effects. Hence, the use of vapour generators that introduce a significantly heated airstream during the decontamination of, for example, the enclosure or packaging in which these cells may be housed is suboptimal. A further example is that the heated vapour stream can create hot-spots on surfaces directly opposite the vapour stream output nozzle. These hot-spots do not allow for the formation of micro-condensation and hence microbial inactivation in these locations can be adversely affected. This is a particular problem in small aseptic chambers that are often used to manipulate CBHTs. In addition, hydrogen peroxide vapour generators designed around the power availability in Europe or Asia may have to operate at lower performance levels for the decontamination of large volumes in other locations due to having insufficient power to heat their vaporiser plates and the incoming carrier air stream sufficiently.

Energy consumption reduction is desirable for equipment users from both cost and environmental perspectives. Energy consumption reduction is also desirable for equipment manufacturers as a reduction in components or sub-assemblies associated with thermal regulation and heating will contribute to lower manufactured costs of equipment, smaller equipment foot-print, simpler controls, easier and cheaper servicing costs, lower shipping costs and improved reliability.

The basis of the development of decontaminant vapour generators stemmed from the requirement to bio-decontaminate aseptic processing environments such as isolators. The methods described in patents WO 01/21223 A1 and US 5906794 reflect this basis, as the methods relate to “closed-loop” systems, whereby a vaporiser unit is connected to a sealed chamber via pipe connections. The theories of Watling apply in this situation, where the carrier gas again must be heated to ensure that the decontaminant remains in the vapour state whilst it is transported along the pipelines from the vapour generator to the target chamber - i.e. the enclosed space. If the temperature of the carrier gas is at a point where it becomes saturated by the vapour it is conveying, the vapour will condense out of the carrier gas onto the surfaces of the pipeline and thus will not be delivered to the desired location.

WO-A-2006/031957 discloses a flash vaporiser which comprises a heating block containing one or more bores to produce a constant flow of evaporated decontaminant used to decontaminate large enclosures or buildings. The bores within the heated block increase in diameter as the hydrogen peroxide travels through the block to account for the increase in expansion as the decontaminant changes from liquid to gas. The flash vaporised decontaminant is combined with a dried and heated carrier airstream in a duct that transfers it to the enclosure to be decontaminated.

US 5906794 discloses a means to decontaminate enclosures involving a single loop configuration, wherein the apparatus has a single pathway comprising a dehumidifier/dryer, a catalyst, a heater and a flash evaporator. Air within the enclosure is prepared and hydrogen peroxide is introduced into the enclosure via the pathway. Air returning to the apparatus from the enclosure is passed through the catalyst and dehumidifier, controlling the concentration of vapour within the enclosure and preventing the formation of micro-condensation.

EP2155267B1 discloses an apparatus similar to that described in EP-A-1487503 characterised in that it has a passageway containing a heater, a flash evaporator and a means to pump air through the passageway. It has a second passageway through which air is moved through and into the enclosure. The outlets of the first and second passageway are separate. Again, EP2155267B1 depends upon the creation of a heated carrier gas (air) stream.

It is an object of this invention to provide a method and apparatus that allows a vapour phase decontaminant to be introduced into an enclosed space using a significantly less energy intensive and simpler to control process. By reducing the energy requirements of such methods and apparatus it is anticipated that the weight, the cost of manufacture, operating efficiency, controls complexity and carbon footprint of such decontamination systems can be significantly reduced. These and other uses, features and advantages of the invention will be apparent to those skilled in the art from the teachings provided herein.

### SUMMARY OF THE INVENTION

The present inventor has identified advantageous configurations of apparatus that enable the use of a non-heated - e.g. ambient temperature - carrier gas within vapour decontamination systems without compromising on decontamination effectiveness.

In a first aspect the invention provides an apparatus for vapour decontamination of an enclosed space, the apparatus comprising:
(a) a reservoir comprising a supply of a liquid decontaminant;
(b) a vaporiser unit in fluid communication with the reservoir, the vaporiser unit comprising,
   a body that defines a lumen that passes along a length of the body, the lumen comprising a first end and a second end, wherein the first end receives liquid decontaminant from the reservoir, and
   a heating element that is configured to deliver sufficient thermal energy to effect vaporisation through rapid boiling of the liquid decontaminant within the lumen so that it exits the lumen via the second end as a vapour;
wherein the exit port is configured to deliver the vapour into a flow of a non-heated carrier gas thereby facilitating distribution of the decontaminant vapour within the enclosed space.

A second aspect of the invention provides a method for decontaminating surfaces within an enclosed space comprising locating an apparatus as disclosed herein within the enclosed space, or within a structure surrounding the enclosed space, creating a decontaminant vapour from a solution of a liquid decontaminant by rapidly boiling the liquid decontaminant within the vaporiser unit of the apparatus, introducing the vaporised decontaminant into the enclosed space until the atmosphere within the enclosed space is saturated with vapour such that the dew-point of the decontaminant is reached and micro-condensation of the decontaminant occurs on surfaces within the enclosed space so as to effect decontamination of the surfaces. The atmosphere within the enclosed space may be at ambient pressure or may be at a negative pressure relative to an external environment, wherein introducing the vaporised decontaminant into the enclosed space comprises introducing the vaporised decontaminant into a non-heated carrier gas stream.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

The present invention can take many forms and arrangement of the steps. One or more embodiments of the invention will now be described, by way of example only and not to be construed as limiting the invention, with reference to the accompanying drawings, in which

FIG. 1 is a drawing of a sealed and enclosed space containing a vapour generator and hydrogen peroxide breakdown scrubbers as described in one embodiment of the present invention;

FIG. 2 is a schematic drawing of one arrangement of apparatus of an embodiment of the invention;

FIG. 3 provides an illustration of one embodiment of a vaporiser unit;

FIG. 4 provides an illustration of one embodiment of an arrangement of multiple vaporiser units within an apparatus;

FIG. 5 provides an illustration of a further embodiment of a possible arrangement of multiple vapour generators within a sealed and enclosed space;

FIG. 6 is a schematic drawing of another arrangement of apparatus of an embodiment of the invention; and

FIG. 7 is a schematic drawing of a distribution header.

### DETAILED DESCRIPTION OF THE INVENTION

The present invention directed, in part, towards a desire to improve upon previous approaches to vapour decontamination methods and apparatus by providing systems that are less demanding in terms of manufacturing resources and energy consumption as well as easier to control. First generation hydrogen peroxide vapour (HPV) technology was developed and commercialized in the late 1990s with using a so-called “single-loop” approach (see US 5906794, mentioned above). This approach involved high energy demand as well as substantial manufacturing complexity. The single-loop approach required the hydrogen peroxide vapour to be injected into an enclosed space, such as a chamber or room, and then broken down, dehumidified and more peroxide added - all in a single circuit. Second generation technology was developed and commercialized in the 2000s using an alternative “dual-loop” technology (see WO0121223A1, mentioned above). Whilst this simplified the technology and reduced the energy demand by splitting the process into two circuits it required the hydrogen peroxide vapour to be injected into a pre-heated air-stream - which consumes significant amounts of energy and requires associated heating and control equipment. As described above, the aim of the current technology is to introduce a vapour phase decontaminant into a chamber or room and have it laid down on all surfaces in a uniform layer a few microns thick through condensation in order to inactivate micro-organisms on the surfaces.

The present invention has sought to further improve upon first and second generation systems, which still represent the industry standard, by providing methods and apparatus that further reduce the energetic demand of the systems and make the systems easier to control and/or integrate into enclosures (e.g. enclosures incorporating robots used for the sterile manufacture of drugs or other advanced therapeutic medicinal products) as well as providing components that are far less complex to manufacture, thereby further simplifying the process.

Prior to setting forth the invention, a number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term “enclosed space” is intended to refer to an enclosed volume of space that is defined by solid boundaries, such as walls, that are relatively impermeable to gas transfer. Suitably the enclosed space is defined within an enclosure, for example, such as a room, vessel, conduit, cupboard, tent or a chamber. The enclosed space will typically comprise an atmosphere as well as one or more windows, ports, doors or other access channels. The enclosed space may be in intermittent or continuous gaseous communication with an external ventilation system such as a heating, ventilation and air conditioning (HVAC) system. The enclosed space may incorporate atmospheric pressure control apparatus in order to generate a negative pressure within it as compared to an external environment.

The term “vapour” is used herein to denote the use of chemical sterilant, such as hydrogen peroxide (H₂O₂) in solution with water. Typically aqueous concentrations of hydrogen peroxide are used of not less than around 27% weight by weight (w/w), and typically between 30%w/w to 35%w/w. However, lower concentrations may also be utilised with corresponding alteration of operating parameters and concomitant reductions in microbial inactivation performance to accommodate any alteration in physical properties of the solution. The aqueous solution is converted to vapour phase typically via a rapid boiling process. In typical use, the hydrogen peroxide vapour is introduced into the atmosphere of an enclosed space continuously until the atmosphere reaches saturation or “dew point” when the vapour may condense on surfaces contained within the enclosed space.

The term “rapid boiling” as used herein refers to a process in which a liquid is exposed to thermal energy over a very short period of time sufficient to achieve full or partial transition to the vapour phase. The term “flash evaporation” conventionally involves depressurisation in combination with phase change, however in the field of vapour decontamination technology the term is used to refer also to the vaporisation of a stream of hydrogen peroxide solution applied under gravity or by way of a pump to a heated surface, typically within a heated airflow. This dropping of liquid peroxide onto a hot plate in order to achieve near instant phase change is more fully described in, for example, US20140037496A1. In contrast, rapid boiling occurs when a supply of hydrogen peroxide solution is introduced into a passageway or conduit which is heated sufficiently to enable progressive rapid boiling of the solution as it progresses through the passageway and exits as a vapour. In embodiments of the present invention, rapid boiling of hydrogen peroxide in aqueous solution occurs prior to combination of the subsequent vapour with a non-heated carrier gas.

The term “vapour generator” is used herein to denote apparatus that houses inter alia one or more of: a supply of hydrogen peroxide; vapour generation systems - such as one or more vaporiser units; transport mechanisms - e.g. one or more pumps - for conveying the hydrogen peroxide from a supply reservoir, through the apparatus and to vent to the atmosphere; one or more sensors; one or more controllers; communication systems to permit operational telemetry and remote control if desired; atmospheric control and distribution mechanisms; and a power supply. The aforementioned list is not limiting and it is appreciated that the apparatus of the invention may further comprise a range of units, modules and mechanisms that may permit for mobile or static installation, as well as devices that permit cooperation or communication with ancillary apparatus including but not limited to: atmospheric control and distribution systems, catalytic scrubbers, irradiation devices, heaters, chillers, refrigeration systems, robotics and automation, autonomous vehicles, process supervisory control and data acquisition systems (e.g. SCADA), and such like. The vapour generator may be comprised within a static unit or comprised within a mobile apparatus. In an embodiment of the invention the mobile apparatus is comprised within a robotic system or autonomous vehicle.

The term “vaporiser” or “vaporiser unit” is used herein to denote a device comprised within or in communication with the vapour generator that effects conversion of aqueous hydrogen peroxide to vapour phase via a rapid boiling step prior to venting the vapour into a carrier gas stream.

The term “carrier gas” is used herein to denote a gas that is used to distribute the hydrogen peroxide from the vapour generator into an atmosphere comprised within an enclosed space. Typically the carrier gas will be relatively inert and will not interact chemically or physically with the hydrogen peroxide vapour. Suitably, the carrier gas is selected from one or more of air (including dehumidified air); nitrogen; argon; and carbon dioxide. The vapour may be applied to the carrier gas via one or more vents or injection ports. Typically, the carrier gas is accelerated into a gas flow or stream that passes through or by such vents or ports.

The term “non-heated” in relation to the carrier gas is used herein to denote that the gas may be employed at a temperature substantially ambient to that of the vapour generator - e.g. the ambient temperature of the enclosed space to which the vapour is to be applied. According to embodiments of the present invention the carrier gas is not subjected to any thermal treatment within the apparatus prior to combination with the hydrogen peroxide vapour. Such a gas may also be referred to as unheated.

Utilising a stream of carrier gas that is unheated and substantially ambient to the enclosed space to which the vapour is applied is desirable not just from an energy saving perspective, but also from a practical point of view. Firstly, a heated stream of carrier gas presents issues for products that display high levels of temperature sensitivity, such as cell or gene-based products. This is becoming a more important issue with increasing investment and research into cell and gene-based therapies, including stem cells, as current decontamination systems can result in temperature increases of up to at least 5° C. in the space to which the decontaminant vapour is applied as well as the formation of local ‘hot-spots’ in the direct line of the heated airstream.

Secondly, current systems employing heated carrier gas streams can encounter issues when decontaminating relatively small chambers due to the introduction of excessive heat. The heated carrier gas causes the temperature of the atmosphere and surfaces to increase, driving away the dew-point of the decontaminant vapour and preventing the laying down of sufficient decontaminant for effective inactivation of micro-organisms. This leads to the requirement to prolong the gassing phase of the decontamination treatment so more decontaminant is introduced into the chamber. However, since the carrier gas is heated, this also introduces more heat into the chamber. In addition to exacerbating the issues described above when dealing with temperature-sensitive products or reagents, the additional heat drives away the dew-point of the decontaminant vapour even more and also provides more heat to surfaces in the chamber. Both of these factors make it harder for the decontaminant to condense out of the carrier gas stream and onto the surfaces, which can result in challenges with the decontamination performance. The use of heated carrier gas streams in small chambers or enclosures therefore leads to a recurring feedback loop which is never able to adequately solve the issues experienced with unsatisfactory decontamination performance.

It is therefore clear that vapour decontamination methods and apparatus would benefit from the use of an unheated stream of carrier gas. However, with current systems, this has proven to be a challenge, with a reduction in the temperature of the carrier gas causing the process to ‘stall’ and have the decontaminant vapour condense out of the carrier gas stream in characteristic puffs of white ‘smoke’ which comprise locally condensed vapour, thereby reducing the effective operation of the system. Operating current systems without any heating of the stream of carrier gas can lead to immediate condensation of the decontaminant, with puddles of liquid decontaminant forming under the apparatus. The embodiments of the present invention aim to address these issues and provide a vapour decontamination system that can operate successfully without requiring the use of a heated stream of carrier gas.

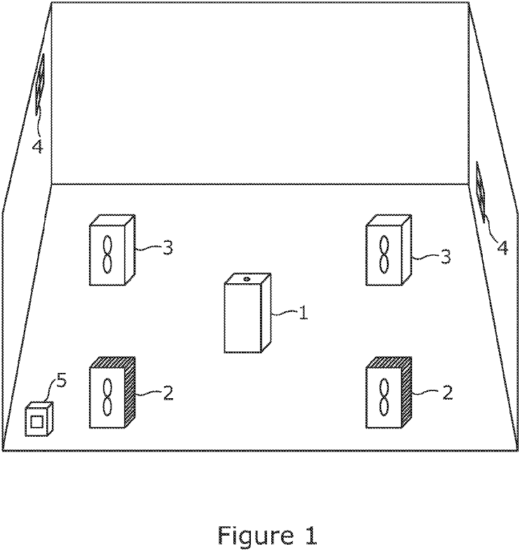

With reference to FIG. 1., an embodiment of the invention provides a method and apparatus wherein a decontaminant vapour generating apparatus (1) is located within an enclosed space defined by a sealed enclosure such as a hospital ward, a laboratory, a pharmaceutical cleanroom, a piece of pharmaceutical apparatus, a food production hall, a fast food restaurant, a hotel room, a public transportation carriage, a compartment, a cabin, a shipping container etc to be decontaminated. The apparatus may be accompanied by devices to aid in the supply of carrier gas as well as distribution of the decontaminant vapour around the enclosure such as fans (3) and catalytic aeration units, scrubbers or other decomposition units (2) to remove the decontaminant vapour at the end of the decontamination process. Alternatively, the decontaminant vapour may be removed using an environmental control system, such as an HVAC system connected to the enclosure, with the decontaminant vapour being exhausted to atmosphere subject to applicable local emissions regulations (where it is broken down to water vapour and oxygen by exposure to UV light from the sun). A catalytic filter may be included in the HVAC filter.

The apparatus (1) may also be accompanied by environmental sensors to determine the efficacy of the decontamination process such as biological indicators, chemical indicators, enzymatic indicators or any other indicator that measures the decontamination process. These indicators (4) are positioned at challenge locations within the enclosed space, such as in corners and behind furniture or installed equipment, or may be placed in locations within the enclosed space where the temperature is identified as being higher - e.g. localised hot-spots where dew formation is least likely to occur. The sensors may also measure the environmental conditions within the enclosed space such as an instrumentation module (5) that can contain one or more sensors such as a humidity sensor, a pressure sensor, a temperature sensor or decontaminant concentration sensor. It is possible to include one or more of these sensors in the vapour generating apparatus (1) as an alternative embodiment of the instrumentation module.

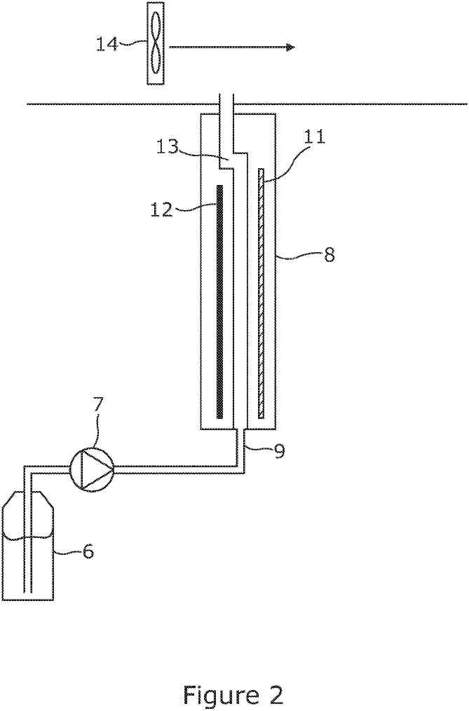

An embodiment of the vapour generating apparatus (1) is shown in FIG. 2. The apparatus comprises a reservoir (6) for the decontaminant. This may be a permanent reservoir, or a reservoir such as a bottle that can be inserted into and removed from the apparatus. The reservoir is in fluid communication with a pump (7), such as a calibrated peristaltic pump, which transports the decontaminant solution from the reservoir (6) to a vaporiser unit (8). An alternative embodiment (not shown) utilises a non-calibrated pump with the reservoir (6) located on a weighing scale or pressure pad in order to determine the rate of delivery of the decontaminant as the reservoir (6) is depleted.

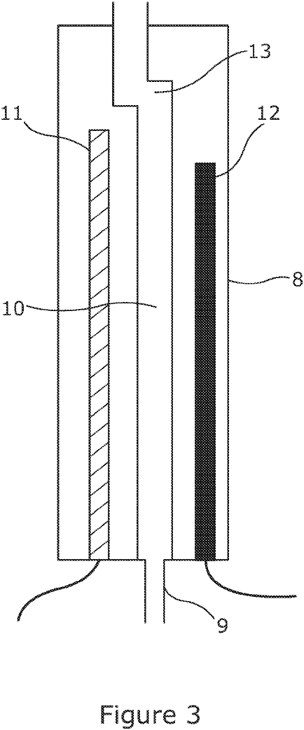

The vaporiser unit (8) comprises a body that is constructed from a thermally conductive material, such as anodised aluminium, stainless steel, etc. The body comprises a hollow lumen (10) in the form of a bore extending longitudinally therethrough that receives liquid decontaminant at a first end and vents vaporised decontaminant from a second end (see FIG. 3.). At least one heating element (11), such as a cartridge heater, is located within the body of the vaporiser to heat the unit. It will be appreciated that alternative heating elements may be selected, including those that may comprise a coil heater, or a ceramic heater. In an alternative embodiment the heating element (11) may be integrated into the vaporiser unit (8) as a cast heating block. In one embodiment, a heating element (11), such as a cartridge heater, is located coaxially with respect to the lumen (10). A temperature sensor (12), such as a temperature probe or thermocouple, may be located within the body of the vaporiser unit (8) facilitating control of the operation of the heating element (11) via an electrical controller located within or in electrical communication with the apparatus (1). The controller may regulate flow of decontaminant to the unit (8), for example by controlling the pump (7) or via another flow regulator mechanism, so as to ensure that an excess of liquid decontaminant is not received. The temperature sensor (12) may be located proximate to the lumen (1) and/or the heating element (11).

The vaporiser unit (8) is heated and controlled to a temperature in excess of the boiling point of the decontaminant solution. The decontaminant solution enters the vaporiser via a port (9) located at the first end of the lumen (10). The temperature of the decontaminant solution is rapidly elevated to boiling point and transitions from liquid to vapour phase, in turn causing gaseous expansion that contributes to accelerated travel along the vaporiser lumen (10) to exit the unit (8) at the second end. The decontaminant is maintained in the vapour phase as it travels along the lumen (10). In an embodiment, the unit (8) further includes a baffle (13) which restricts any non-vaporised decontaminant droplets from continuing along and exiting the unit (8). Non-vapour droplets remain within the main section of the lumen (10) and are subsequently vaporised and may then pass through the baffle unhindered. In an alternative embodiment, the unit (8) is angled substantially vertically or slightly off vertical, for example between about 1 and about 89° of the horizontal, allowing for the use of gravity to segregate the vaporised and non-vaporised decontaminant.

In the embodiments shown in FIGS. 2 and 3 the vaporiser unit (8) can be manufactured relatively simply from an aluminium, or other metal or metal alloy, workpiece that is machined to include coaxially aligned drill holes that define the lumen (10), and which accommodate the heating element (11) and temperature sensor (12). The baffle (13) can be defined by intersecting offset drill holes that cooperate to define a kink or dogleg in the lumen (10).

The heated and expanding decontaminant vapour is vented from the second end of the lumen (10). An impeller (14), such as a fan, can be used to mix the vapour with the atmosphere and distribute it within the enclosed space. In an alternative embodiment, the vapour may exit the vaporiser unit (8) into a conduit. A fan or other form of impeller (14) may be located within or proximate to the conduit. In one embodiment, the apparatus (1) may comprise ducting whose ends are open to the enclosed space and in which the impeller (14) is located. The conduit may vent the vapour into the ducting to enable mixing of the vapour with the carrier gas, such as air, prior to dispersal in the enclosed space. It is apparent that there is no requirement for preheating or other thermal treatment of the carrier gas within the apparatus (1) prior to combination with the decontaminant vapour either in the conduit, ducting or in the enclosed space.

In use, at a point dictated by the starting humidity and temperature of the air within the enclosed space and the partial vapour pressure of the decontaminant, the atmosphere within the enclosure becomes saturated and no longer capable of supporting further addition of decontaminant vapour. At this point, termed the “dew-point”, the decontaminant starts to lay down onto the surfaces within the enclosure as micro-condensation. The micro-condensation is not always visible to the naked eye and droplets can be less than 3 microns in diameter. Addition of further vapour to the enclosure, results in a thickening of the condensation on the surface. Micro-organisms on the surfaces within the enclosure are subject to the decontaminant micro-condensation, inactivating them (see Unger-Bimczok & Kottke (2008) J. Pharm Innov. 3:123-133). The required contact time of the decontaminant micro-condensation with the micro-organism is dependent on the physiological structure of the micro-organism and its relative resistance to inactivation by decontaminants such as hydrogen peroxide. Once sufficient contact time has elapsed, the decontaminant decomposition units (2) are activated removing the decontaminant and rendering the enclosed space fully decontaminated and safe for reoccupation and use. The vapour decontamination cycle may be repeated to provide increased assurance of microbial inactivation, if required.

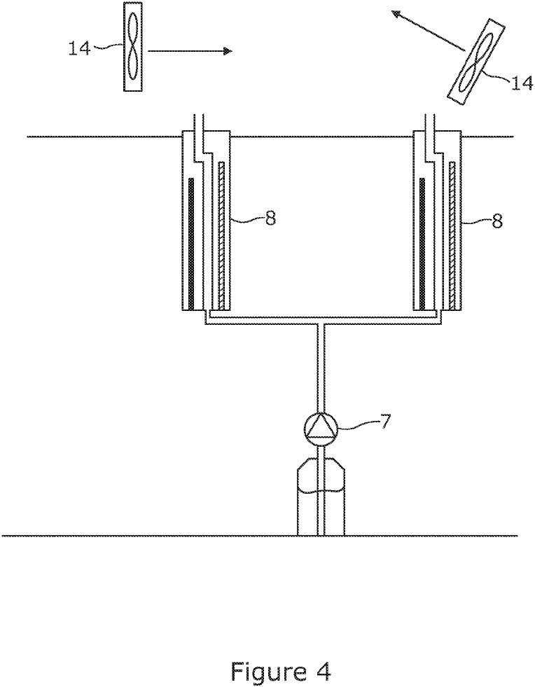

FIG. 4. presents an alternative embodiment of the invention in which the apparatus (1) comprises multiple vaporiser units (8) to increase the injection rate of the decontaminant into the enclosure. The pump (7) provides and distributes decontaminant solution from a reservoir to two or more vaporiser units (8) simultaneously. One or more impeller units (14) cooperate to distribute vapour within a carrier gas stream within an enclosed space.

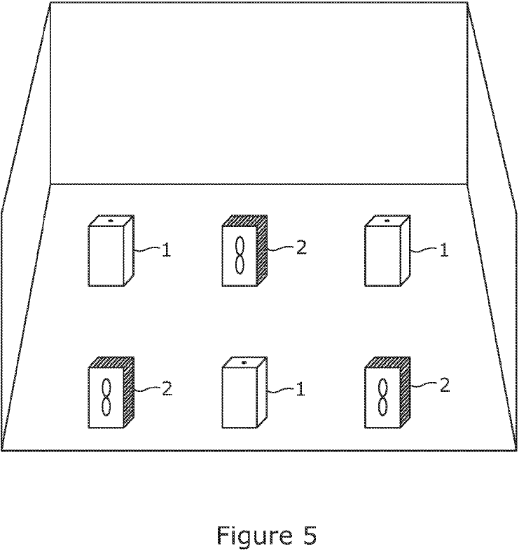

An alternative configuration is presented in FIG. 5. The illustration provides an example of multiple vaporiser apparatus (1) distributed within an enclosed space, with multiple catalytic aeration units (2).

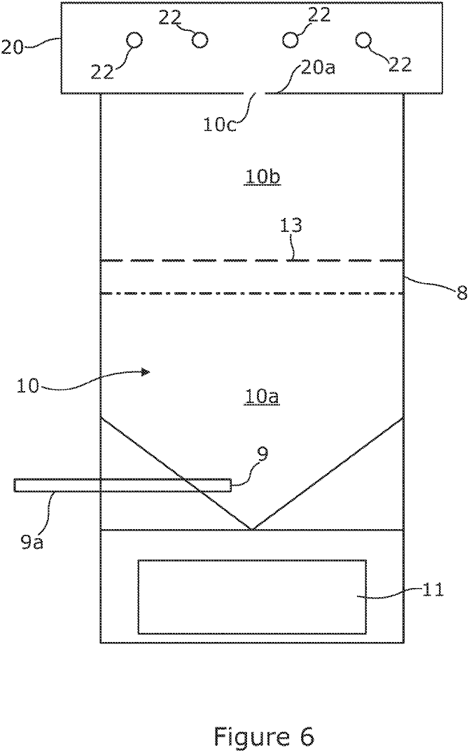

An alternative embodiment of the invention is shown in FIG. 6, which shows a schematic representation of parts of a vaporiser apparatus (1). It should be appreciated that the arrangement shown in FIG. 6 is purely schematic and is shown to aid clarity of the description only. In FIG. 6, like reference numerals are used to denote like features to the embodiments described above. FIG. 6 shows a vaporiser unit (8), which comprises a lumen (10) for vaporising liquid decontaminant, and a distribution header (20). The lumen (10) is in fluid communication with a port (9) at a first end of the lumen (10), which opens into a conduit (9a). Liquid decontaminant enters the lumen (10) via the conduit (9a) and the port (9) via the action of a pump (7, not shown in FIG. 6).

As with previous embodiments, the walls of the vaporiser unit (8), and consequently the lumen (10), are heated by a heating element (11) embedded in the vaporiser unit (8) to enable the liquid decontaminant that enters the lumen (10) to be vaporised. As before, a temperature sensor (12, not shown in FIG. 6), such as a temperature probe or thermocouple may be located within the body of the vaporiser unit (8) to facilitate control of the operation of the heating element (11) via an electrical controller located within or in in electrical communication with the apparatus (1). The lumen (10) is divided into a first, rapid boiling chamber (10a) and second, vapour re-heating chamber (10b) by a vapour sieve (13), such that the first and second chambers (10a, 10b) are in fluid communication with each other.

The heating of the lumen (10) by the heating element (11) ensures vaporisation of the liquid decontaminant by rapid boiling while it is in the first chamber (10a). As discussed above, rapid boiling involves a liquid being exposed to sufficient thermal energy over a short period of time to ensure full or partial transition to the vapour phase. As such, when in the first chamber (10a), the liquid decontaminant is heated under the action of the heating element (11) to rapidly elevate its temperature by rapid boiling, so it transitions from the liquid to vapour phase. The rapid expansion of the liquid decontaminant as it vaporises creates a pressure gradient that drives the vapour from the first chamber (10a) towards the second chamber (10b).

The vapour sieve (13) works to prevent liquid decontaminant from entering the second chamber (10b). As in the embodiments described above, the vapour sieve (13) may take the form of a baffle, or an arrangement of baffles, but it may equally take the form of a physical sieve to prevent the passage of liquid decontaminant between the first and second chambers. Through the action of the vapour sieve (13), non-vaporised decontaminant remains in the first chamber (10a), where it is eventually vaporised and passes through to the second chamber (10b). It should be noted, however, that the residency time of the decontaminant within both the first and second chambers (10a, 10b) is extremely short, such that there is effectively no breakdown of the decontaminant when travelling through the lumen (10). This is especially important when using a volatile decontaminant, such as hydrogen peroxide.

The second chamber (10b) is heated by the action of the heating element (11) and keeps the temperature of the vaporised decontaminant high to avoid so-called ‘stalling’ of the vapour, where it condenses. As a further mode of protection against the passage of liquid decontaminant through the lumen (10), the vaporiser unit (8) can be arranged so that the lumen (10) is aligned vertically or angled to the horizontal. In this way, gravity provides an extra force to resist the passage of liquid decontaminant through the system. The lumen arrangement described above may also be incorporated into the embodiment of the vaporiser apparatus (1) shown in FIGS. 1 to 5.

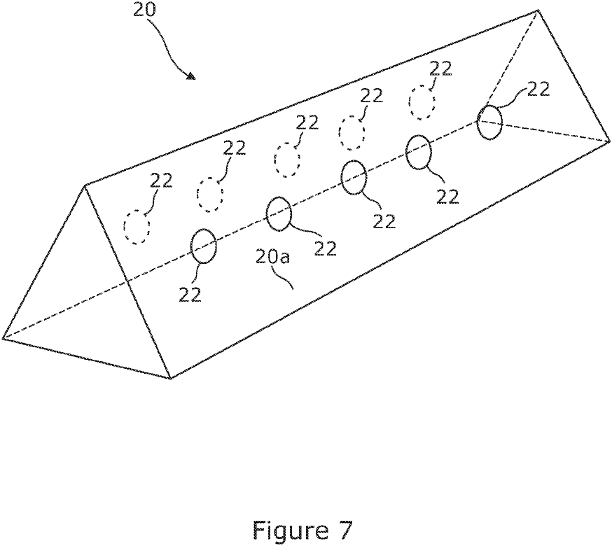

The distribution header (20) is, shown schematically in FIG. 7, in fluid communication with the second chamber (10b). In use, the vaporised decontaminant leaves the second chamber (10b) via an exit port (10c) at a second end of the lumen (10) and enters the distribution header (20). The distribution header (20) provides a third chamber for the vaporised decontaminant, greater in volume than both the first and second chambers (10a, 10b), from where controlled venting of the decontaminant can be effected. To this end, the distribution header (20) comprises one or more distribution outlets (22) for vapour decontaminant egress. The distribution header (20) is also thermally coupled to the heating element (11) to facilitate heating of a bottom surface (20a) of the distribution header (20) to help ensure that vaporised decontaminant contained therein remains in the vapour phase. The distribution header is also heated by virtue of the hot vapor phase decontaminant exiting the lumen (10) into the distribution header (20). In an alternative embodiment, the heat from the heating element (11) may be insufficient to heat the distribution header (20), or the distribution header may be thermally insulated from the heating element (11) such that the distribution header (20) is primarily, or only, heated by the hot vapour phase decontaminant passing through it from the lumen (10).

The distribution header (20) shown in FIG. 6 is integral with the vaporiser unit (8). In an alternative embodiment, the distribution header (20) may be a separate component which is in fluidic communication with the exit port (10c) of the lumen (10) of FIG. 6. Such a separate distribution header (20) may also be used with the vaporiser unit (8) of FIGS. 1 to 5 if desired.. In each case, there may be thermal contact between the bottom surface (20a) of the distribution header (20) and the body of the vaporiser unit (8), to help avoid stalling and condensation of the vapour contained therein. In an alternative embodiment, the distribution header (20) may be substantially or completely thermally decoupled from the heating element (11) such that the distribution header (20) is partailly, or only, heated by the hot vapour phase decontaminant passing through it from the lumen (10) in use.

The aim of the distribution header (20) is to provide greater control of the vapour that is vented into the carrier gas stream, such that the carrier gas stream does not need to be heated to avoid the vapour stalling. The vaporised decontaminant upon leaving the second chamber (10b) has high thermal energy and also a high kinetic energy, driven by the large pressure gradient arising from the rapid boiling of the decontaminant in the first chamber. The distribution head (20), having a greater volume then the first or second chambers (10a, 10b), acts to partially neutralise the effect of the pressure gradient, thereby reducing the kinetic energy of the vaporised decontaminant while keeping the thermal energy high through thermal contact with the heating element (11). It should be understood that only the base of the distribution header (20) is provided with thermal energy, thus negligible heat transfer occurs into the carrier gas. The distribution header (20) can also be insulated to prevent unwanted heating of the carrier gas. By venting the vaporised decontaminant with lower kinetic energy, the decontaminant is more easily entrained within the flow of the carrier gas stream, enabling the carrier gas to perform its function without requiring additional heating.

Depending on the number and position of the impellers (14), the number and position of the distribution outlets (22) on the surface of the distribution header (20) may vary in order to optimise take-up of the vaporised decontaminant by the carrier gas stream. In a certain embodiment, the distribution header (20) takes the form of a triangular prism, as seen in FIG. 7. In this embodiment, the distribution outlets (22) are arranged on angled face of the prism. The inventor has found that positioning the distribution outlets (22) such that that the vaporised decontaminant exits the distribution header (20) at an angle to the flow of the carrier gas in this way improves entrainment of the vapour by the carrier gas stream, further helping the avoidance of stalling in the vapour.

In certain embodiments, the impeller (14) may be positioned below the vaporiser assembly (1), such that the carrier gas stream is generated upstream of the distribution outlets (22). Such a configuration avoids interference with the vaporised decontaminant from cavitational effects arising from pressure differentials at tips of the blades of the impeller (14), which can trigger stalling of the vapour and failure of the system, with droplets of condensed decontaminant being produced.

The vaporiser apparatus (1) may additionally comprise a ducting system to guide the flow of the carrier gas before and after entrainment of the vaporised decontaminant. The ducting system may comprise separator panels (not shown) above the distribution header (20) to separate the vaporised decontaminant emanating from each distribution outlet (22). This ensures even take-up of vapour across the carrier gas stream and more even distribution of decontaminant around the enclosure.

The ducting system of the vaporiser apparatus (1) may further comprise guide panels (not shown) to guide the carrier gas stream and the entrained vaporised decontaminant as it exits the vaporiser and assist with the distribution of the carrier gas and vaporised decontaminant throughout the enclosure. In certain embodiments, the guide panels comprise a substantially horizontal cover panel for the vaporiser apparatus (1) in combination with angled panels to define a vent through which the carrier gas and vaporised decontaminant can leave the apparatus (1) and enter the enclosure. The angled panels may be contiguous with the separator panels.

The embodiments described above allow the utilisation of unheated carrier gas by minimising the risk of stalling of the decontaminant vapour. As discussed above, the use of an unheated carrier gas stream presents solutions to the issues experienced with the use of current systems employing heated carrier gas streams in small spaces and for temperature sensitive products.

However, the use of an unheated carrier gas stream has other advantages. By obviating the need to heat the carrier gas, the apparatus (1) of the invention requires a significantly lower power draw, consumes significantly less energy than systems where the carrier gas is heated and can be designed with smaller volume and lower weight. Without the need to heat the carrier gas, the apparatus (1) provides faster operation with smaller and lighter equipment and shorter decontaminating cycles, as there is no need to get the equipment needed to heat the carrier gas up to temperature. Taken together, these advantages lead to a significantly reduced carbon footprint.

With only one heater, electrical control of the apparatus is significantly simplified. In addition, the simplicity of design of the vaporiser apparatus allows for significant reductions in the complexity of manufacture compared to prior art systems, reducing manufacturing costs and improving reliability. These benefits are therefore also significant contributors to the enhanced environmental credentials of the methods and apparatus described.

The aforementioned embodiments are not intended to be limiting with respect to the scope of the appended claims, which follow. The choice of decontaminant, its rate of vaporisation, the specific design of the vaporiser unit and the quantity of carrier gas is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

What is claimed is:

1-29. (canceled)

30. An apparatus for vapour decontamination of an enclosed space, the apparatus comprising:
(a) a reservoir comprising a supply of a liquid decontaminant;
(b) a vaporiser unit in fluid communication with the reservoir, the vaporiser unit comprising,
a body that defines a lumen that passes along a length of the body, the lumen comprising a first end and a second end, wherein the first end receives liquid decontaminant from the reservoir, and
a heating element that is configured to deliver sufficient thermal energy to effect vaporisation through rapid boiling of the liquid decontaminant within the lumen so that it exits the lumen via an exit port at the second end of the lumen as a vapour;
wherein the exit port is configured to deliver the vapour into a flow of a non-heated carrier gas, thereby facilitating distribution of the decontaminant vapour within the enclosed space.

31. The apparatus of claim 30, wherein the vaporiser unit comprises a distribution header in fluid communication with the exit port of the lumen, the distribution header comprising,
a chamber for receiving the vapour, and
one or more distribution outlets;
such that the decontaminant vapour is delivered into the carrier gas via the distribution outlets.

32. The apparatus of claim 31, wherein the distribution header defines a volume greater than that of the lumen.

33. The apparatus of claim 31, wherein the chamber of the distribution header is heated.

34. The apparatus of claim 31, wherein the one or more distribution outlets are located on a face of the distribution header that is arranged at an angle to the flow of the carrier gas.

35. The apparatus of claim 30, wherein the vaporiser unit comprises a vapour sieve that separates the lumen into a first chamber and a second chamber, wherein the vapour sieve is configured to prevent the passage of liquid decontaminant from the first chamber to the second chamber.

36. The apparatus of claim 30, wherein the apparatus comprises ducting to direct the flow of the carrier gas.

37. The apparatus of claim 36, wherein the ducting comprises separator panels configured to separate the vapour emanating from each distribution outlet.

38. The apparatus of claim 30, wherein the apparatus further comprises:
(c) an impeller that provides the flow of carrier gas.

39. The apparatus of claim 38, wherein the impeller is located below the vaporiser unit.

40. The apparatus of claim 38, wherein the impeller is located within the ducting.

41. The apparatus of claim 30, wherein the heating element comprises a cartridge heater.

42. The apparatus of claim 30, wherein the apparatus further comprises a transport mechanism for transporting the liquid decontaminant to the vaporiser unit, wherein the transport mechanism comprises a pump.

43. The apparatus of claim 30, wherein the apparatus comprises a plurality of vaporiser units.

44. The apparatus of claim 30, wherein the liquid decontaminant comprises an aqueous solution of hydrogen peroxide.

45. A method for decontaminating surfaces within an enclosed space comprising:
locating an apparatus of claim 30 within the enclosed space, or within a structure surrounding the enclosed space;
creating a decontaminant vapour from a solution of a liquid decontaminant by rapidly boiling the liquid decontaminant within the vaporiser unit comprised of the apparatus;
introducing the vaporised decontaminant into the enclosed space until the atmosphere within the enclosed space is saturated with vapour such that the dew-point of the decontaminant is reached and micro-condensation of the decontaminant occurs on surfaces within the enclosed space so as to effect decontamination of the surfaces,
wherein introducing the vaporised decontaminant into the enclosed space comprises introducing the vaporised decontaminant into a non-heated carrier gas stream.

46. The method of claim 45, wherein after the decontaminant has resided on the surface for sufficient time to effect the required decontamination, the decontaminant is removed from the sealed enclosure.

47. The method of claim 45, wherein the enclosed space is maintained at a lower atmospheric pressure than the surrounding environment.

48. The method of claim 45, further comprising repositioning the apparatus within the enclosed space.
